(19) United States
(12) Patent Application Publication
(10) Pub. No.: US 2020/0098294 A1
(43) Pub. Date: Mar. 26, 2020

(54) Vision adjustable screen system

(71) Applicant: Ahmad Abo Alsheghri, Montreal (CA)

(72) Inventor: Ahmad abo Alsheghri, Montreal (CA)

(21) Appl. No.: 16/578,520

(22) Filed: Sep. 23, 2019

(30) Foreign Application Priority Data
Sep. 25, 2018 (CA) 3018464

(51) Int. Cl. G09G 3/00; G02B 3/00

(52) U.S. Cl. CPC G09G 3/003; A61B 3/0041; G02B 3/005

(57) ABSTRACT

Vision adjustable screen system to enable people with different eyes diseases to read, watch and use their devices like, mobile, laptop, desktop, I pad, TV, Etc. Without glasses by adjusting the screen to suit their eyes disease, this screen consist of adjustable two layers screen with electronic components in between with integrated software in the screen or in the device, other solution to use vision mobile application for mobiles and on applicable devices

Since I am accountant spending a lot of time on my computer using my glasses which annoying me I thought there must be a solution to do my job without using my glasses and her I got the idea specially I used to work as CNC programmer manufacturing complicated parts for all kinds of machines for five years.

My invention is a System of two layers adjustable screens (digital screen, 3D screen)with electronic components in between them with integrated software in the screen or in the device to enable people with different eyes diseases to read, watch and use their devices, like (mobile, laptop, desktop, I pad, TV, Etc.), Without glasses by adjusting the setting in the system to suit their eyes disease.

The screen will scan their eyes and adjust automatically to their eyes disease or they insert the vision test information in the system setting or they move the adjustable tools in the system until they get the right sitting

The screen could be used as a separate device to be installed on devices already in use and to be sold to the devices manufacturers to be installed on their new production instead of the old screen as special devices for people with eyes disease where they can switch from regular mode to special mode that suit their eyes disease.

The screen will be in different sizes, shapes and components to suit all kinds of devices

The user will be able to choose his eyes disease from a menu then choose the specific Degree that suits his eyes disease, eyes disease like blurred vision, strabismus, anisometropia, Etc.

The screen will be designed to reduce the effect of the radiation coming out of it, which harms the eyes so people can use their devices for long time with less risk harming their eyes.

Other solution to Make vision mobile application that scan the eyes and adjust the text according to all eyes vision problems or insert the vision test information in the application, where no more need to glasses to read or watch, this system consist of digital screen and application.

It will function as designed screen and graphic card with new specifications with required electronic circuits and new programed software or mobile application to control the user input data to give order through the electronic circuits to the graphic card and the screen to give the required output display which suits the user eyes problems

Example: My Vision adjustable screen system for smart TV where the TV will have setting for eyes disease where the user can set it to their eyes needs as special mode and can switch back to regular mode if he is watching the TV with somebody else.

We can add this software to windows and mac software in laptop and desktop and in mobiles and IPAD for apple and android.

The embodiments of the invention in which an exclusive property or privilege is claimed are defined as follows:

1. Vision adjustable Screen system for people with eyes disease
A separate adjustable screen to be installed on used devices like mobile, laptop, desktop, I pad, TVs, etc. with built in eyes protector to help people with eyes disease to use their devices without need to glasses where they can adjust the screen to suit their eyes problems, and to be sold to the devices manufacturers to install it on their new production instead of the old screen as special devices for people with eyes disease where they can switch from regular mode to special mode that suit their eyes disease, this system consist of adjustable two layers screen with electronic components in between and integrated software in the screen
It will function as designed screen and graphic card with new specifications with required electronic circuits and new programed software to control the user input data to give order through the electronic circuits to the graphic card and the screen to give the required output display which suits the user eyes problems

2. Vision adjustable Screen system for people with eyes disease
A separate adjustable screen to be installed on used devices like mobile, laptop, desktop, I pad TVs, etc. To help people with eyes disease to use their devices without need to glasses where they can adjust the screen to suit their eyes disease, and to be sold to the devices manufacturers to install it on their new production instead of the old screen as special devices for people with eyes disease where they can switch from regular mode to special mode that suit their eyes disease this system consist of special screen with electronic components in between and software to be installed in the device with eyes protector
It will function as designed screen and graphic card with new specifications with required electronic circuits and new programed software to control the user input data to give order through the electronic circuits to the graphic card and the screen to give the required output display which suits the user eyes problems

3. Vision adjustable Screen system for people with eyes disease
A separate adjustable digital screen to be installed on used devices like mobile, laptop, desktop, I pad TVs, etc. To help people with eyes disease to use their devices without need to glasses where they can adjust the screen to suit their eyes disease, and to be sold to the devices manufacturers to install it on their new production instead of the old screen as special devices for people with eyes disease where they can switch from regular mode to special mode fit their eyes disease this system consist of adjustable digital screen and integrated software in the screen
It will function as designed screen and graphic card with new specifications with required electronic circuits and new programed software to control the user input data to give order through the electronic circuits to the graphic card and the screen to give the required output display which suits the user eyes problems

4. Vision adjustable Screen system for people with eyes disease
A separate adjustable digital screen to be installed on used devices like mobile, laptop, desktop, I pad TVs, etc. To help people with eyes disease to use their devices without need to glasses where they can adjust the screen to suit their eyes disease and to be sold to the devices manufacturers to install it on their new production instead of the old screen as special devices for people with eyes disease where they can switch from regular mode to special mode fit their eyes disease, this system consist of digital screen and software to be installed in the device
It will function as designed screen and graphic card with new specifications with required electronic circuits and new programed software to control the user input data to give order through the electronic circuits to the graphic card and the screen to give the required output display which suits the user eyes problems

5. Vision adjustable Screen system for people with eyes disease
3D adjustable screen to be used by manufacturers of the devices like mobile, laptop, desktop, I pad, TVs, etc. For people with eyes disease to use their devices without need to glasses where they can adjust the screen to suit their eyes disease this system consist of 3d adjustable screen with electronic components and integrated software in the device
It will function as designed screen and graphic card with new specifications with required electronic circuits and new programed software to control the user input data to give order through the electronic circuits to the graphic card and the screen to give the required output display which suits the user eyes problems

6. Vision adjustable Screen system for people with eyes disease
Digital adjustable screen to be used by manufacturers of the devices like mobile, laptop, desktop, I pad, TVs, etc. For people with eyes disease to use their devices without need to glasses where they can adjust the screen to suit their eyes disease this system consist of digital adjustable screen with electronic components and integrated software in the device
It will function as designed screen and graphic card with new specifications with required electronic circuits and new programed software to control the user input data to give order through the electronic circuits to the graphic card and the screen to give the required output display which suits the user eyes problems

7. Vision adjustable Screen system for people with eyes disease
Special adjustable screen to be used by manufacturers of the devices like mobile, laptop, desktop, I pad, TVs, etc. For people with eyes disease to use their devices without need to glasses where they can adjust the screen to suit their eyes disease this system consist of special adjustable screen with electronic components and integrated software in the device
It will function as designed screen and graphic card with new specifications with required electronic circuits and new programed software to control the user input data to give order through the electronic circuits to the graphic card and the screen to give the required output display which suits the user eyes problems

8. Vision System for people with eyes disease
Mobile Application that scan the eyes and adjust the text according to all eyes vision problems or insert the vision test information in the application manually, it will help people with eyes disease to use their devices like mobile, laptop, desktop, I pad, TVs, etc. without need to glasses where they can adjust the application to suit their eyes disease this system consist of 3d adjustable screen with Application
It will function as designed screen and graphic card with new specifications with required electronic circuits and new programed mobile application to control the user input data to give order through the electronic circuits to the graphic card and the screen to give the required output display which suits the user eyes problems

9. Vision System for people with eyes disease
Mobile Application that scan the eyes and adjust the text according to all eyes vision problems or insert the vision test information in the application manually, it will help people with eyes disease to use their devices like mobile, laptop, desktop, I pad, TVs, etc. without need to glasses where they can adjust the application to suit their eyes disease this system consist of adjustable digital screen and Application
It will function as designed screen and graphic card with new specifications with required electronic circuits and new programed mobile application to control the user input data to give order through the electronic circuits to the graphic card and the screen to give the required output display which suits the user eyes problems

10. Vision System for people with eyes disease
Mobile Application that scan the eyes and adjust the text according to all eyes vision problems or insert the vision test information in the application manually, it will help people with eyes disease to use their devices like mobile, laptop, desktop, I pad, TVs, etc. Without need to glasses where they can adjust the application to suit their eyes disease this system consist of special screen with Application
It will function as designed screen and graphic card with new specifications with required electronic circuits and new programed mobile application to control the user input data to give order through the electronic circuits to the graphic card and the screen to give the required output display which suits the user eyes problems

11. Vision System for people with eyes disease
Mobile Application that scan the eyes and adjust the text according to all eyes vision problems or insert the vision test information in the application manually, it will help people with eyes disease to use their devices like mobile, laptop, desktop, I pad, TVs, etc. Without need to glasses where they can adjust the application to suit their eyes disease this system consist of digital screen and Application
It will function as designed screen and graphic card with new specifications with required electronic circuits and new programed mobile application to control the user input data to give order through the electronic circuits to the graphic card and the screen to give the required output display which suits the user eyes problems
